(19) United States
(12) Patent Application Publication
(10) Pub. No.: US 2025/0325621 A1
(43) Pub. Date: Oct. 23, 2025

(54) COMPLEX EXTRACT OF SOUR CHERRY AND PROCESS FOR THE PREPARATION OF SUCH EXTRACT

(71) Applicant: DEBRECENI EGYETEM, Debrecen (HU)

(72) Inventor: Judit REMENYIK, Debrecen (HU)

(21) Appl. No.: 18/870,929

(22) PCT Filed: May 30, 2023
(86) PCT No.: PCT/HU2023/050030

§ 371 Date: Dec. 2, 2024

(30) Foreign Application Priority Data
May 30, 2022 (HU) P2200188

(51) Int. Cl. A61K 36/736; A23L 33/105; A61K 8/9789; A61K 9/48; A61P 1/16; A61Q 11/00

(52) U.S. Cl. CPC A61K 36/736; A23L 33/105; A61K 8/9789; A61K 9/48; A61P 1/16; A61Q 11/00; A61K 2236/333

(57) ABSTRACT

The subject of the invention is a complex extract made from sour cherry, characterized in that it
has a viscosity at 20° C. of about 2500-3800 mPa·s
has a density of 1.30-1.85 g/cm³
dissolves well in water and in alcohol,
is substantially free of fibres
   and advantageously
has a deep burgundy colour and sour cherry smell.

A further subject of the invention is a process for the preparation of such extract from sour cherry, wherein extraction is carried out in several steps with water-alcohol solvent mixtures of increasing alcohol content, and the concentrates obtained at the end of each extraction step are added to the eluate obtained in the next extraction step before its evaporation.

The invention also relates to preparations containing the complex sour cherry extract.

The application is the national stage of International Application PCT/HU2023/0050030, filed May 30, 2023.

### THE FIELD OF THE INVENTION

The subject of the invention is a complex extract of sour cherry and a process for the preparation of such extract from sour cherry. The extract with a complex composition according to the invention contains a wide spectrum of components of the original fruit and has a high anthocyanin content. The invention also relates to preparations containing the complex sour cherry extract.

### STATE OF THE ART

There is a growing demand in the food industry to produce natural, local, healthy, additive-free and safe foods. In Hungary, thanks to decades of sour cherry breeding and research work, we have an outstanding range of varieties, which is unique not only in Europe, but also in the world. Thanks to the geographical and climatic characteristics of our country, the sour cherry varieties selected through landscape selection have outstanding inner content values, especially regarding their anthocyanin content. There are many references in the international literature that examine the beneficial effects of anthocyanins from red berries in in vivo experiments, or in vitro models. Nearly 500 types of anthocyanins have been described in various plants (cranberries, raspberries, strawberries, grapes, etc.), however, the study of sour cherry anthocyanins is still in the initial stages. The main active components of the sour cherry, cyanidin-3-O-monoglucoside and cyanidin-3-O-rutinoside, are present in very high concentrations in the fresh fruit.

The anthocyanins of our cultivated sour cherry varieties are special organic compounds that cannot be efficiently produced synthetically. Both domestic and international research groups worked on the laboratory production of anthocyanins, as there is great interest in them due to their positive physiological effects. The reason for the failure of the production is that the synthetic production of the flavonium cation is already a challenge during the preparative work, but the selective binding of the sugar aglycone has not yet been solved either, even though this ensures the biological activity. Even the complicated protecting group strategies used during the synthesis did not bring a resounding success for the high-yield, economical production of anthocyanins. The chemical structure of anthocyanins is very sensitive to pH changes, since the sugar is easily hydrolysed, and thus the molecule loses its stability and effect.

Thanks to the climatic characteristics of our country and the unique range of varieties, an average of 150-200 mg/100 g of anthocyanin accumulates in Hungarian sour cherry varieties, which is unique in the world. Thus, the efficient extraction of natural anthocyanins synthesized in sour cherries is of great interest.

In recent years, there has been an increasing interest in phytotherapeutic possibilities, which has led to the use of different extraction technologies to produce extracts of natural origin and free of various solvent residues. The food, the pharmaceutical and the cosmetic industries have also developed procedures. Plant extracts and essential oils produced mainly by traditional extraction methods are used by the cosmetics industry as fragrances or as anti-inflammatory agents in the preparations. These are usually produced by steam distillation processes. The pharmaceutical industry produces extracts for the preparation of various dietary supplements and medicinal preparations, the food industry produces extracts for using as spice extracts, natural antioxidants, or natural dyes, focusing on certain families of compounds. The technologies used are solid-liquid extractions, liquid-liquid extractions, and supercritical carbon dioxide extractions (Zhang et al. “Techniques for extraction and isolation of natural products: a comprehensive review” Chin. Med. (2018) 13:20).

Commercial plant extracts with a high anthocyanin content in powder-form are typically produced using a supercritical process, which is selective for a group of compounds. In powder-form extracts anthocyanin is bound to a carrier, e.g. to cyclodextrin, maltodextrin, cellulose (pl. EP 2545787 A1).

Liquid extracts with high anthocyanin content are typically tinctures with a significant alcohol content, as anthocyanins do not dissolve well in water.

There are also products on the market with high antioxidant capacity, produced by concentrating fruit juices. An example of such an approach is patent application No. P1400413, which describes a jam-like product made by concentrating selected fruit juices and adding pectin and vitamins. It goes without saying that in this case the quality and active ingredient content of the fruit juices determines the amount of active ingredients in the product. No measure (extraction method) is described in the document to extract the valuable components found in the fruit juice.

In the article of Homoki et al. (2018): Sour cherry extract inhibits human salivary α-amylase and growth of Streptococcus mutans (a pilot clinical study). FOOD AND FUNCTION, 9 (7). pp. 4008-4016, the salivary alpha-amylase-inhibiting effect of a chewing gum containing sour cherry extract is described, however, the sour cherry extract used in the chewing gum is not characterized and its production is not described.

Patent description No. GB1235379 describes a process for extracting anthocyanins from fruits. During the process, the fruit (e.g. cherries, blackcurrants, etc.) is pressed, the fibre is macerated, e.g. with a water-alcohol mixture (specific solvent composition is not mentioned in the document), and the resulting liquid is combined with the squeezed fruit juice and concentrated. This concentrate is evaporated to dryness at a higher temperature and then further purified to extract the anthocyanins.

Patent description HU 204 984 A describes a process for the preparation of alcohol-free fruit and vegetable products by processing with ethyl alcohol, followed by dealcoholisation before use. According to the document, the plant used as a raw material is prepared in a known manner, the ethyl alcohol content of the prepared plant material is set to 15-45% by volume, the resulting, possibly fibrous juice is left to rest for at least 3 weeks, and then the solid material and the eluate is separated, the alcoholic eluate is stored for as long as desired, and then, before the desired use, in a film distillation apparatus, using a dynamic carbon dioxide and/or inert gas atmosphere, and if desired, absorbing CO₂ or inert gas in the dosing tank as well, after preheating to a temperature of 50-100° C., at a pressure of 6.4-26.6 kPa at a temperature of 40-80° C. the alcohol is distilled from it by flash distillation, and the low-alcohol product obtained as a bottom product is, if desired, processed using known food industry processes.

Patent description WO2004089891A1 describes a process for isolating antioxidants from plant waste by extraction with polar solvents, preferably water or a mixture of water and ethanol, optionally followed by evaporation. The isolate contains polyphenols, anthocyanins, sugars and has an antioxidant capacity.

None of the above three documents describes extraction with different solvent mixtures.

None of the above documents describes extraction with solvent mixtures of increasing alcohol content and decreasing polarity.

The article of Andrea Nemes et al, Determination of Flavonoid and Proanthocyanidin Profile of Hungarian Sour Cherry, Molecules 2018, 23, 3278 relates to the determination of the active ingredient profile of fresh sour cherries/sour cherry varieties; production of sour cherry extract is not described in it. They describe the use of increasing ethanol content for the extraction (water:ethanol 50:50 then pure ethanol), but no solvent with a high water content is used and the first extract is not combined with the subsequent eluate. They do not produce a complex extract; in fact, the aim of the work is different: the extraction of certain active ingredients and the determination of the active ingredient profile of the sour cherry varieties.

According to US 2009/0011056 A1, active ingredients are extracted from cranberries. Here too, increasing ethanol content (80 and 95% ethanol) for the extraction is described, and after that a group of active ingredients is relatively cleanly obtained by chromatographic purification (92.7% of the extract obtained with 80% ethanol is made up of 3 specified active ingredients, and 50% of the extract obtained by a second extraction of the remaining raw material with 95% ethanol consists of a single active ingredient, resveratrol). The use of a solvent with a high water content and the merging of the extracts during the process are not included, and a no complex extract is produced. None of the processes described in any of the above documents is suitable to produce an extract with a complex composition from sour cherries, which contains a wide spectrum of the components of the original fruit, preferably essentially its entire sugar and acid content (which are highly polar compounds), as well as the less polar bioactive components, especially anthocyanins and/or proanthocyanidins.

### The Problem to be Solved

There is therefore a need for a process that can effectively extract soluble substances from cherries, including valuable bioactive substances such as anthocyanins, and where the activity of the bioactive substances in the product obtained by the process is preserved for a long time.

### SUMMARY OF THE INVENTION

We have found that with a process in which extraction is carried out in several steps with water-alcohol solvent mixtures of increasing alcohol content, and the concentrates obtained at the end of each extraction step are added to the eluate obtained in the next extraction step before its evaporation, it is possible to produce a complex extract that contains a wide spectrum of the components of the original fruit, preferably essentially its entire sugar and acid content, as well as the less polar bioactive components, especially anthocyanins and/or proanthocyanidins. The activity of the bioactive substances in the obtained complex extract remains for a long time.

Without wishing to be bound by any theory, we believe that the substances extracted during the first extraction step with a solvent mixture with a high water content (which are mainly polar compounds, including sugars, fruit acids) exert a stabilizing effect. In their presence, the eluate can be evaporated without considerable decomposition of the biologically active substances, which are sensitive to heat and light, and the finished product, the complex extract, can also be stored without preservatives or refrigeration, even for several years. In other words, the fact that in the first step extraction is carried out with a solvent mixture with a high water content and the material extracted thereby are carried forward in the process as mentioned, ensures the stability of the sensitive biologically active substances both during the process and in the product.

The invention relates to a process for the preparation of a complex extract from sour cherry, the process comprising
a first extraction cycle, wherein
   sour cherry is extracted with a water:ethanol solvent mixture,
   the extraction mixture is separated into its phases, thus obtaining an eluate and a phase comprising residual solid material,
   the eluate is evaporated, thus obtaining a first concentrate,
one or more further extraction cycle(s), wherein
   the fraction comprising residual solid material, obtained in the previous extraction cycle, is extracted with a water:ethanol mixture, whose ethanol content is higher than that of the water-ethanol mixture used in the previous extraction step,
   the extraction mixture is separated into its phases, thus obtaining an eluate and a phase comprising residual solid material,
   the thus obtained eluate is combined with the concentrate obtained in the evaporation step of the previous extraction cycle, and this mixture is evaporated thus obtaining another concentrate from the current extraction cycle,
wherein
   the first extraction is carried out using a 90:10-65:35 water:ethanol solvent mixture, and
   the last extraction is carried out using a 50:50-0:100 water:ethanol solvent mixture, wherein the complex extract is obtained as the concentrate from the last extraction cycle.

Thus, each concentrate from the given extraction cycle is specific to the given cycle, and its sequence number corresponds to the sequence number of the given cycle, and also includes the concentrate from the previous extraction cycle.

In one embodiment of the invention two extraction cycles are carried out:
the first extraction is carried out using a 90:10-65:35 water:ethanol solvent mixture, and
the second extraction is carried out using a 50:50-30:70 water:ethanol solvent mixture.

In another embodiment of the invention two extraction cycles are carried out:
the first extraction is carried out using a 90:10-65:35 water:ethanol solvent mixture, and the second extraction is carried out using a 35:65-0:100 water:ethanol solvent mixture.

In a further embodiment of the invention
the first extraction is carried out using a 90:10-65:35 water:ethanol solvent mixture, and
the second extraction is carried out using a 65:35-35:65 water:ethanol solvent mixture, and a third extraction is also carried out using a 35:65-0:100 water:ethanol solvent mixture.

In a further embodiment of the invention
the first extraction is carried out using a 85:15-75:25 water:ethanol solvent mixture, and
the second extraction is carried out using a 60:40-40:60 water:ethanol solvent mixture, and
a third extraction is also carried out using a 30:70-0:100 water:ethanol solvent mixture.

The first extraction step is carried out within a time period of at most 24 hours, preferably within at most 12 hours (that is, we leave the sour cherries in the extractant for this amount of time before separating them into phases).

The further extraction steps are carried out within a time period of at most 12 hours, preferably within at most 6 hours.

During the extraction, the temperature is preferably at most 45° C., more preferably about 20-40° C., for example about 20-28° C.; conveniently, we operate at ambient temperature.

The individual extraction steps are typically performed over a period of about 1-12 hours.

Evaporation is typically carried out at about 25-40° C., using reduced pressure.

Preferably, the ratio of the raw material (prepared sour cherries) and the solvent mixture is about 0.5-3 L solvent mixture/1 kg of raw material, preferably 0.75-2 L of solvent mixture/1 kg of raw material.

To separate the extraction mixture into its phases, we can use e.g. filtration, sedimentation or centrifugation.

In one embodiment of the invention, the separation of the extraction mixture into its phases is carried out by filtration, and the aperture size of the filter used in the last filtration step is at most 0.01 cm, preferably 0.005 cm, more preferably 0.0005 cm.

The invention also relates to the complex extract obtainable by the above-described process.

The invention also relates to a complex extract characterized in that it
has a viscosity at 20° C. of about 2500-3800 mPa·s, preferably measured with a Viscolead One type viscometer,
has a density of 1.30-1.85 g/cm³
dissolves well in water and in alcohol, and
is substantially free of fibres.

The complex extract has a deep burgundy colour and sour cherry smell. It has the consistency of honey, and it is thickly flowing.

In one embodiment, the complex extract has a water-soluble antioxidant capacity (ACW) of about 0.21-0.6 μg/mg extract, the total amount of phenolic compounds is about 0.06-0.2 μg/mg extract.

In another embodiment, the complex extract has a proanthocyanidin content of about 0.3-1.3 g/100 g extract.

In a further embodiment, the complex extract is further characterized in that it comprises the following components: cyanidin-3-O-rutinoside, cyanidin-3-O-glucoside and cyanidin-3-O-glucosyl-rutinoside,
their total amount is about 700 mg-2500 mg/100 g extract, typically 700 mg-1100 mg/100 g extract, and their relative ratio is as follows:
   cyanidin-3-O-glucosyl-rutinoside: about 0.7-1.3 mass %
   cyanidin-3-O-rutinoside: about 65-73 mass %
   cyanidin-3-O-glucoside: about 27-32 mass %.

In one embodiment, the complex extract comprises:
glucose, fructose and saccharose, wherein the relative ratio of the individual components within their total amount is:
   glucose: 35-60 mass %,
   fructose: 30-55 mass %,
   saccharose: 5-15 mass %, and
malic acid and citric acid, wherein the relative ratio of the individual components within their total amount is:
   malic acid: 85-98 mass %,
   citric acid: 2-15 mass %.

The amount of the mentioned acids and sugars can be determined e.g. with a kit Sucrose/D-Glucose/D-Fructose (Art. No. 10716260035), L-Malic acid (Art. No. 10139068035) and Citric acid (Art. No. 10139076035) manufactured by Boehringer Mannheim/R-Biopharm.

The above-defined complex extract can be advantageously prepared by the method according to the invention.

A further object of the invention is a composition, preferably a dietary supplement, food for special medical purposes or medical device, comprising a complex extract according to the invention.

The composition according to the invention optionally comprises the complex extract formulated in capsules.

A further object of the invention is a food composition intended for human consumption, comprising the complex extract according to the invention and other food ingredients.

A further object of the invention is a feed composition for animals, comprising the complex extract according to the invention and other feed ingredients.

A further object of the invention is a composition, preferably a dietary supplement, food for special medical purposes or medical device comprising the complex extract as defined above, for use in preventing tooth decay; preventing or reducing inflammations, e.g. colitis, prostatitis; reducing pain and muscle pain caused by osteoarthritis; regulating carbohydrate metabolism, including lowering blood sugar; reducing the feeling of hunger, preventing obesity; preventing non-alcoholic fatty liver disease or promoting liver regeneration processes in case of an already established disease; preventing cancer, or inhibiting of the growth of tumour cells in the case of an already established tumour.

A further object of the invention is the use of the complex extract as defined above for the preparation of a composition, preferably a dietary supplement, food for special medical purposes or medical device, for preventing tooth decay; preventing or reducing inflammations, e.g. colitis, prostatitis; reducing pain and muscle pain caused by osteoarthritis; regulating carbohydrate metabolism, including lowering blood sugar; reducing the feeling of hunger, preventing obesity; preventing non-alcoholic fatty liver disease or promoting liver regeneration processes in case of an already established disease; preventing cancer, or inhibiting of the growth of tumour cells in the case of an already established tumour.

A further object of the invention is a method of preventing tooth decay; preventing or reducing inflammations, e.g. colitis, prostatitis; reducing pain and muscle pain caused by osteoarthritis; regulating carbohydrate metabolism, including lowering blood sugar; reducing the feeling of hunger, preventing obesity; preventing non-alcoholic fatty liver disease or promoting liver regeneration processes in case of an already established disease; preventing cancer, or inhibiting of the growth of tumour cells in the case of an already established tumour, wherein the complex extract defined above is administered to a person.

### Definition of Terms and Abbreviations Used in the Description

In the description, “complex extract” is understood to mean an extract that does not only contain one or a few groups of active ingredients, but contains most of the soluble components of the sour cherry, preferably essentially all of them, separated from the insoluble fibres, in a concentrated form. The complex extract is therefore characterized by the fact that besides the active components, e.g. anthocyanins and proanthocyanidins, it also contains the sugar and acid components that are characteristic of sour cherries.

In the description, “eluate” is understood to mean the liquid phase obtained after the separation of the solid material after the individual extraction steps.

In the description, “fibre” is understood to mean the insoluble solid material remaining after extraction. Most of it is made up of polysaccharides, but it also contains other insoluble residues (e.g. cell wall, other components that make up cells).

In the description, the term “substantially fibre-free” is understood to mean that the extract contains essentially no fibres larger than about 0.01 cm, preferably about 0.005 cm, more preferably about 0.0005 cm. The size of the fibres remaining in the extract is determined by the aperture size of the filter used, if filtration is used to separate the solid and liquid phases.

The appearance of the concentrated product (complex extract) is purely transparent, not cloudy. This appearance indicates that the fibres forming a coarse dispersed system have been removed from the product. However, when the complex extract is diluted, these fibres below the specified limit size, which are mainly oligosaccharides, may become visible. However, the amount of these fibres is much smaller than the fibre content of the whole fruit (no more than about one-tenth of the fibre content of the original fruit, typically no more than 2, preferably no more than 1 g/100 g of extract), and they do not significantly reduce the absorption of the other components.

The ratios given in the description for the solvent mixtures, unless otherwise indicated, are to be understood as volume ratios, given for 96% ethanol by volume.

In the description, in connection with specifying the ratios, ethanol is meant to be an ethanol of 96 volume %, so a composition with a ratio of 0:100 water:ethanol is also meant to be ethanol of 96 volume %. Preferably the alcohol of 96 volume % is at least food grade ethanol.

In the description, in relation to the composition of the solvent mixtures, the term “higher ethanol content” means that the solvent mixture contains a higher proportion of ethanol than in the previous solvent mixture. The difference between the ethanol content of two successive solvent mixtures is preferably at least 10% by volume.

### BRIEF DESCRIPTION OF THE DRAWINGS

FIGS. 1 to 5 show the results of the clinical blood chemistry tests performed in the animal experiments according to Example 7, and FIG. 6 shows the results of the gene expression test. The date of the test is indicated above the diagrams (in the case of blood tests). The examined groups of animals were, respectively, the following:
C—control group receiving normal rabbit food,
C+ant—group receiving sour cherry extract treatment in addition to normal rabbit food,
HC—atherogenic diet group,
HC+ant—group receiving sour cherry extract treatment in addition to an atherogenic diet.

In the figures, the stars show the level of significance between the individual results according to one-way ANOVA: * means p<0.05, ** means p<0.005, *** means p<0.0002 and **** means p<0.0001.

FIG. 1 shows plasma cholesterol levels.

FIG. 2 shows plasma AST levels.

FIG. 3 shows plasma ALT levels.

FIG. 4 shows plasma GGT levels.

FIG. 5 shows plasma ALP levels.

FIG. 6 shows the relative expression of cystathion gamma-lyase on the left and cystathion beta-synthase on the right.

### DETAILED DESCRIPTION OF THE INVENTION

Our aim was to develop a sequence of extraction steps that can be used to extract a wide range (spectrum) of the components that make up the total soluble dry matter content of the sour cherry, including its sugar and acid content (which are highly polar compounds), as well as the less polar bioactive components, especially anthocyanins and/or proanthocyanidins. For this, we searched for solvent combinations that can be used in the food industry or can be completely removed with a simple technological step, e.g. by distillation.

We have found that by using mixtures of ethanol with decreasing polarity (increasingly higher ethanol content) this aim can be reached.

However, when evaporating the eluate obtained in the second, third extraction (using solvent mixtures with higher ethanol content), we experienced significant decomposition of the active components.

Surprisingly, we have found that the evaporation can be performed without significant decomposition, if the concentrate obtained from the first extraction (containing mainly sugars and acids) is added to the eluate obtained from the second extraction, and likewise, in case of performing further steps, the concentrate obtained from the previous extraction (containing the concentrate obtained optionally after several extraction) is added to the eluate obtained from the subsequent extraction.

Furthermore, we have found that the physical and chemical stability of the most important active components of the sour cherry, the anthocyanins, is preserved in the complex extract obtained in this way without the use of stabilizers or other additives.

In preparation for the procedure, the sour cherry is cleaned before extraction. Cleaning may include e.g. sorting, de-stemming, washing, removing the seeds. We can use fresh or frozen our cherries, or the pulp obtained from them, as a base material.

There is usually no need to chop the sour cherries beforehand, because according to our experience, the mixing used during the extraction ensures a sufficient amount of fragmentation.

The first step is performed with a water:ethanol mixture with high water content (preferably, the volume ratio of water:ethanol is in the range of about 90:10-65:35). The extraction mixture is separated into its phases, thus obtaining an eluate and a residual solid material. The eluate is evaporated, thus obtaining a first concentrate, the residual solid material that remains after the filtration is extracted with a water:ethanol solvent mixture, whose ethanol content is higher than that of the water-ethanol mixture used in the previous extraction step, and optionally, this sequence of operations can be repeated several times. The evaporation of the respective eluates is performed as described later.

The extraction is performed in several (at least two) steps, using water:ethanol mixtures with increasing ethanol content, and in the last step, a solvent mixture with high ethanol content (with a ratio of 50:50 or a higher ethanol content, for example with a water:ethanol ratio of at least 35:62, 25:75 or 10:90) or pure ethanol (of 96%) is used.

The extraction can be performed for example in two, three, four or five steps. At least two steps are necessary in order to efficiently extract ingredients of different polarity from the base material. In practice, we have found that typically 3 extraction steps are enough to ensure substantially complete extraction of the soluble components of the base material. Performing more than three steps generally does not significantly improve the overall efficiency of the process, therefore, preferably three extraction steps are performed during the process, using solvents with increasing ethanol content.

Using solvent mixtures with different composition ensures the extraction of substantially all soluble components from the fruit, starting from the polar, water-soluble compounds to the less polar compounds that are soluble in alcohol.

If the extraction is carried out in two steps, the applied solvent mixtures can have the following composition:
1st extraction: 90:10-65:35 water:ethanol
2nd extraction: 50:50-0:100 water:ethanol.

Also in the case of two steps, it is necessary to perform the first extraction with a solvent mixture with high water content (90:10-65:35 water:ethanol), as a mixture with such a composition ensures the extraction of sugars and acids, and the concentrate of this fraction is able to stabilize the eluate obtained using a mixture with higher ethanol content during its evaporation. The composition of the second solvent mixture is chosen depending on whether the aim is obtaining a higher anthocyanin content or a higher proanthocyanidin content. A medium alcohol content favours the extraction of the anthocyanins (although a part of the proanthocyanidins is also extracted), while a high alcohol content favours the extraction of the proanthocyanidins (although a part of the anthocyanins is also extracted in this case). Whichever composition is used, a wider spectrum of ingredients can be extracted in just two steps than what is possible with methods known from the state of the art, therefore the final concentrate obtained in this way is also considered a complex extract according to the invention, which, in addition to sugars and acids, contains a significant amount of active components.

If the extraction is carried out in three steps, the applied solvent mixtures can have for example the following composition:
1st extraction: 90:10-65:35 water:ethanol
2nd extraction: 65:35-35:65 water:ethanol
3rd extraction: 35:65-0:100 water:ethanol.

We have found that during the first extraction essentially the entire amount of sugars and fruit acids, as well as a small amount of colouring matter is extracted. The second extraction mainly enables the extraction of anthocyanins. During the third extraction with a solvent with high alcohol content, an additional amount of anthocyanins is dissolved, and with this composition the largest amount of proanthocyanidins is dissolved.

The optimal water:ethanol ratio in each step depends on the type of sour cherry used and other parameters of the process, and can be determined by the person skilled in the art.

The difference between the ethanol content of the solvent mixtures used consecutively is preferably at least 10% by volume and at most 90% by volume, for example 10, 15, 20, 25, 30, 35, 40 or 50% by volume. The person skilled in the art is able to determine the optimal difference depending on the number of the steps applied and the aim to be reached. Obviously, when performing fewer extraction steps, applying relatively larger differences is advisable and permissible in order to ensure sufficiently different polarity for the extraction of the different substances, while in case of more extraction steps smaller differences can be applied. For example, if two extraction steps are used, the alcohol content of the second solvent mixture is greater preferably by at least 15% by volume, more preferably by at least 30% by volume, for example, by 40, 50 or 60% by volume, and even a difference of 80-90% by volume is permissible. In case of three steps, there is preferably at least a 10% by volume difference, for example about 15, 20, 25, 30% by volume, between the ethanol content of successively used solvent mixtures.

During the extraction, the mixture is preferably stirred. In the case of sour cherry varieties with softer pulp, it is sufficient to stir the extraction mixture from time to time during the extraction. A too strong agitation will “smear” the fruit, which can greatly complicate the separation of the liquid phase later in the process.

During extraction, the temperature is at most 45° C., preferably about 20-40° C., for example about 20-28° C.; ideally, we operate at ambient temperature (in each extraction step). Increasing the temperature above about 40-45° C. is not beneficial due to heat-sensitive bioactive substances, and cooling is not favourable in terms of extraction efficiency.

In one embodiment, the duration of each extraction step is about 1-12 hours, for example 1-6 hours.

The amount of solvent for the extraction can be determined by the person skilled in the art. On the one hand, care must be taken to ensure that the solvent completely covers the raw material. From the point of view of the efficiency of the extraction, a larger amount of solvent is more favourable, but too much solvent increases the evaporation time, which is not beneficial from the point of view of sensitive compounds. Considering the above, in a preferred embodiment, the ratio of the raw material (prepared sour cherry) and the solvent mixture in the extraction mixture is about 0.75-2 L solvent mixture/1 kg of raw material.

The extraction mixture is then subjected to a separation procedure to separate the solid phase from the liquid phase. For this, we can use e.g. filtration, sedimentation or centrifugation. The aim is to remove the fibre content as efficiently as possible, as fibre can adversely affect the absorption of certain active ingredients. In one embodiment, filtration is performed, preferably by applying an increasingly dense filter after each extraction step. The aperture diameter (D) of the applied filters can range from about 0.1 cm to about 0.0005 cm. As a result of the filtration, the product will have a honey-like clean consistency.

The eluate obtained after extraction and separation is evaporated. After the individual extraction and separation (e.g. filtering) steps, the evaporation is carried out in the following way. The eluate obtained after the first extraction is evaporated, thereby a first concentrate is obtained. (Conveniently, during the evaporation of the individual eluates, we start and, optionally, perform the next extraction and separation step.) The first concentrate is then combined with the eluate obtained after the second extraction, and this mixture is concentrated again, thereby obtaining a second concentrate, which contains all materials that were extracted from the sour cherry by the first two extraction steps. In case a third extraction is also performed, the eluate obtained after the third extraction is combined with the second concentrate, and this mixture is concentrated again, thereby obtaining a third concentrate, which contains all materials that were extracted from the sour cherry by the first three extraction steps. In the case of performing more extraction steps, the eluate obtained from the respective extraction step is combined with the concentrate obtained from the previous extraction step, and this mixture is concentrated. The concentrate obtained after the last evaporation contains all materials that were extracted from the sour cherry by all extraction steps.

In the description, the product obtained by the last evaporation is called complex extract or extract according to the invention.

We have found that adding the first concentrate to the eluate of the second extraction before its evaporation is of key importance. When evaporating the eluate of the second or third extraction alone (see the comparative examples), considerable decomposition was observed. Without wishing to be bound by any theory, we believe that the materials extracted during the first extraction step (which are mainly polar components, including sugars and fruit acids), exert a stabilizing effect. In their presence, the eluate can be evaporated without notable decomposition of the heat- and light-sensitive biologically active components, and the finished product, the complex extract can be stored for several years without preservatives or refrigeration.

Most of the fibre remaining after the last extraction is a non-starch oligosaccharide that can be used as a prebiotic. The fibre left over from sour cherries is particularly rich in fructo- or in arabino-oligosaccharides.

Evaporation can be carried out in a manner known to the person skilled in the art, for example with a standard evaporation device. Evaporation is preferably carried out at about 25-40° C. (water bath temperature), using reduced pressure (e.g. 150-10 mbar). The leaving solvent is preferably led through a cooling tower, where the cooling medium can be e.g. water or an aqueous solution of ethylene glycol (in the case of closed cooling system), and the temperature of the coolant can be e.g. about 0-10° C. In this way, the solvent used for extraction is essentially recovered. Ethanol can be distilled from the recovered water-alcohol mixture and reused.

Evaporation is preferably carried out in the first and optionally other intermediate steps until a viscosity of at least about 1000 mPa·s, preferably at least 2000 mPa·s is reached, even more preferably it is continued essentially until no more water escapes from the concentrate. The advantage of greater evaporation is, on the one hand, that the stabilizing effect of the concentrate is greater and, on the other hand, it does not significantly increase the volume upon addition to the eluate of the subsequent extraction.

The final evaporation is essentially continued until no more water escapes from the concentrate. To monitor this, we can also measure e.g. the viscosity. In one embodiment, evaporation is continued until the viscosity of the evaporated concentrate reaches a value of about 2500-3800 mPa·s, preferably about 2800-3800 mPa·s, more preferably about 3000-3800 mPa·s, at 20° C.

The complex extract obtained by the process of the invention is a thick, viscous liquid (having a honey-like texture). The density of the product is typically about 1.30-1.85, preferably about 1.35-1.85 g/cm³. The viscosity of the product measured with a Viscolead One type viscometer at 20° C. is about 2500-3800 mPa·s, at 40° C. about 300-700 mPa·s, at 5° C. about 6000-7500 mPa·s. This thick, viscous texture is formed without adding thickeners, and indicates the high sugar content of the product, which originates exclusively from the sour cherry used as raw material.

The extract according to the invention has a smell, taste, and colour characteristic to sour cherry (but it is darker and more concentrated).

The pH value of the extract according to the invention largely depends on the type of sour cherry, it is usually about 2.8-4.0, typically about 3.0-3.8. The pH is measured with a pH meter suitable for measuring high-density food products. We can use, for example, a pH meter of type AD 111-IJ44C.

The extract according to the invention is substantially fibre-free, so it has a purely transparent appearance, it is not cloudy. The fibre content of the extract according to the invention is typically no more than about 2 g/100 g of extract, preferably no more than 1 g/100 g of extract. The size of the fibres remaining in the extract is at most about 0.01 cm, preferably at most about 0.005 cm, even more preferably at most about 0.0005 cm. In a preferred embodiment, the fibre content of the extract is at most 1 g/100 g of extract, and the size of the fibres remaining in the extract is at most about 0.005 cm.

The free water content (water activity) of the extract according to the invention is very low, essentially all free water is extracted during the process by evaporation. The water remaining in the extract is in a bound form, which the microorganisms cannot utilize.

According to our experience, the water cannot be completely removed from the extract by standard evaporation or lyophilisation (this indicates that the water remaining in the product is in a bound form); the extract cannot be made into a powder form. As a result of forced lyophilisation, it can be evaporated to dryness, but due to the large specific surfaces formed, contact with air is sufficient for it to clump back.

With the process, the biological matrix of the original fruit is preserved in the complex extract (essentially, we only separate all other soluble components from the fibre).

The ratio of the components in the extract according to the invention therefore corresponds to the ratios found in the starting sour cherry variety. The product can therefore—in addition to the typical active components—be characterized e.g. with the quality and proportions of the sugars and fruit acids found in it.

The microbiological activity (germ count) of the extract is essentially “zero”, measured by the method according to the MSZ EN ISO 4833:2003 standard, and by the method according to the MSZ ISO 7954 standard, there are no detectable mesophilic aerobic microbes or fungus or mould in it.

The extract can be stored for years without preservatives or refrigeration. Preferably, the product can be kept for at least 2 or 3 years, very preferably for at least 4 or 5 years. We currently have a 4-year-old sample that has not lost its quality (see example 5).

With the process, the active ingredients can be extracted in a very good proportion. The important active ingredients of sour cherries are anthocyanins, but in addition to them, precursor compounds of anthocyanins are also present, such as flavonoids, gallo-, ellagitannins, condensed tannins, other polyphenols, minerals, vitamins, and trace elements.

The fact that the active ingredients are essentially fibre-free in the product increases the absorption of certain active ingredients, since the polysaccharides that make up the fibre are capable of forming various secondary bonds, thus binding many compounds.

The remaining fibre is white and essentially no longer contains colouring matters.

The complex extract according to the invention, as a food, ensures access to all active components of sour cherry even outside the ripening season, moreover in a concentrated form and by ensuring a more efficient absorption.

The extract according to the invention can be used in various forms.

The extract according to the invention has a high enjoyment value in itself, so it can be consumed as food, but it is also suitable for further processing, or certain active ingredients/groups of active ingredients can be extracted from it in pure form (e.g. by supercritical extraction, chromatography).

If the fruit raw material is certified “organic”, the process is suitable to produce an organic final product. The extract according to the invention contains neither added sugar nor other additives (e.g. added pectin or carrier material, e.g. cyclodextrin, maltodextrin or the like), and it is non-toxic.

More specifically, the extract according to the invention can be used on its own as a food, dietary supplement, food for special medical purposes or as a medical device, but it is also suitable for further processing, for example it can be added to food for human consumption, functional food, or animal feed, thereby increasing the inner content value of the food or feed, especially its anthocyanin content and thus its antioxidant capacity. Due to its colouring components, the extract according to the invention can also be used to colour food, such as drinks.

According to one embodiment, other food ingredients are also found in the food products or food for special medical purposes.

According to a further embodiment, food products or food for special medical purposes also contain excipients acceptable in food industry or from a nutritional point of view.

More specifically, the extract according to the invention can be consumed alone as a syrup-like product with a consistency similar to honey. In order to achieve precise dosing, it can be packaged e.g. in single-dose sachets, or it can even be encapsulated using capsule cases suitable for encapsulating water-containing substances.

The extract according to the invention can be added to other foods. It can be combined particularly well with confectionery products, dairy products, soft drinks, and spirits. For example, it can be used to make candy, gum, chewing gum, chocolate, but it can also be added to dairy products, e.g. it can also be used for cottage cheese or yogurt preparations, or in baked goods.

The extract according to the invention can also be mixed with drinks, for example milk-based drinks, soft drinks, spirits.

The complex extract can be dosed as a dietary supplement or as a medical device in tablets or capsules.

According to one embodiment, dietary supplements or medical devices also contain other excipients that are acceptable in such formulations.

The extract according to the invention can also be added as an additive to feed for animals, especially farm animals with a single-cavity stomach, e.g. poultry, thereby improving the antioxidant status of animals.

As a result of the process according to the invention, the biological substances are present in a higher concentration in the extract than in the starting fruit, so their favourable physiological effect is increased. With the process, the components present in small amounts in the fresh fruit can be enriched to a dose that is already effective.

Based on the effects documented in the literature, the sour cherry extract according to the invention and the products containing it are useful, among others for preventing tooth decay; preventing or reducing inflammations, e.g. colitis, prostatitis; reducing pain and muscle pain caused by osteoarthritis; regulating carbohydrate metabolism, including lowering blood sugar; reducing the feeling of hunger, preventing obesity; preventing cancer, or inhibiting of the growth of tumour cells in the case of an already established tumour. We have also demonstrated that the sour cherry extract according to the invention and the preparations containing it can be used to prevent non-alcoholic fatty liver disease or to promote the regeneration processes of the liver in the case of an already established disease.

If certain biologically active ingredients are to be used in an even higher concentration, the extract according to the invention can be the starting material for processes suitable for further enrichment, e.g. separation procedures (e.g. column chromatography separation), so it is even possible to isolate certain active substances/groups of active substances.

During the process according to the invention, we do not use any substance that could contaminate the product. The ethanol used during extraction is essentially completely removed during evaporation. The ethanol content of the extract according to the invention is no more than 0.5 mg/100 g of extract.

During the process, no hazardous waste or unusable by-products are formed. The ethanol used for extraction can be recovered and reused by capturing the solvent (water and ethanol) leaving during evaporation, and distilling it. The remaining fibre can be used as a prebiotic.

Surprisingly, we have found that the extract according to the invention can be stored for several years without preservatives or refrigeration. Without wishing to be bound by any theory, we believe that it is owing to the fact that the biological matrix of the original fruit is preserved in the complex extract (essentially, we only separate all other soluble components from the fibre), and this complex biological system ensures the stability of the chemical structures. The low water activity of the extract according to the invention and the antimicrobial components naturally present in the sour cherry can also contribute to the fact that the extract represents an unfavourable environment for the growth of microbes. The alcohol used during the process has a germicidal effect on bacteria and fungi that may be present in the original fruit or on its surface.

The extract according to the invention therefore allows access to the full inner content value of sour cherry (anthocyanins, flavanols, etc.) even outside the ripening season, moreover in a concentrated form. (In the case of traditional preservation methods, e.g. making syrups, jams, compotes, the inner content value is often significantly reduced, and the extracts made with known methods do not contain all the valuable ingredients, they typically only contain one group of active ingredients). This is also a significant advantage because sour cherries have a short ripening period and a poor shelf life. The combined presence of different components is also beneficial as, by strengthening each other's effect, they often exert a more favourable physiological effect than the isolated components.

The active ingredient content and antioxidant capacity of the extract basically depends on the active ingredient content of the starting material, which depends on the variety of sour cherry, the growing conditions, and the degree of ripeness of the fruit.

The extract is generally characterized by that it has a water-soluble antioxidant capacity (ACW) of about 0.21-0.6 μg/mg extract, the total amount of phenolic compounds is about 0.06-0.2 μg/mg extract, and it has a proanthocyanidin content of about 0.3-1.3 g/100 g extract, measured by the methods described below.

The extracts made from each variety can be characterized, for example, by the anthocyanins characteristic of the variety and also by the amount of their precursors. The most characteristic anthocyanins of cherries are in general cyanidin-3-O-rutinoside, cyanidin-3-O-glucoside and cyanidin-3-O-glucosyl-rutinoside. The total amount of these in the extract is usually about 700 mg-2500 mg, typically about 700 mg-1100 mg, and their typical relative ratio is as follows:
cyanidin-3-O-glucosyl-rutinoside: about 0.7-1.3%
cyanidin-3-O-rutinoside: about 65-73%
cyanidin-3-O-glucoside: about 27-32%.

Sugars and fruit acids are also characteristic components. Typically, in sour cherries glucose can be found in the largest amount, there is a slightly smaller amount of fructose (in the case of some varieties, the amount of fructose is higher), and saccharose in a much smaller amount than the two types of sugar mentioned. Among the acids, the amount of malic acid is the largest, the amount of citric acid is about an order of magnitude smaller than malic acid. These proportions essentially persist in the sour cherry extract according to the invention. The proportions depend largely on the type of sour cherry, the growing conditions, and the degree of ripeness of the fruit, but typically fall within the following range:
the ratio of the individual components within the total amount of glucose, fructose and saccharose is:
   glucose: 35-60 mass %
   fructose: 30-55 mass %
   saccharose: 5-15 mass %
the ratio of the individual components within the total amount of malic acid and citric acid is:
   malic acid: 85-98 mass %
   citric acid: 2-15 mass %.

The following non-limiting examples serve to illustrate the invention.

The equipment and procedures used in the examples were as follows.

The extractions were carried out in a vessel equipped with a stirrer.

The filtration was carried out with standard pharmaceutical sieves; the aperture size used is specified for each step.

The refractive index was measured with a Schmidt Haensch #16004 refractometer.

Evaporation was carried out with a Heildolph P/N:501-23000-00-5 rotavapor device.

Viscosity was determined with a Viscolead One type viscometer.

The pH was measured using an AD 111-IJ44C type pH meter.

Sugars and acids were determined using Sucrose/D-Glucose/D-Fructose (Art. No. 10716260035) and L-Malic acid (Art. No. 10139068035) and Citric acid (Art. No. 10139076035) kits, manufactured by Boehringer Mannheim/R-Biopharm.

For UHPLC-MS analyses the following device was used: Hitachi, Autosampler 6270, Diode array detector 6430, Binary pump 6170, Column thermostat 6310. The column used was: PHENOMENEX LC Column KINETEX 2.6 μm XB-C18, 100 Å, 150×4.6 mm.

The antioxidant capacity was determined using the following methods:

Antioxidant capacity measurement based on DPPH (1,1-diphenyl-2-picrylhydrazyl) radical binding: Blois, M. S. (1958): Antioxidant determination by the use of stable free radicals. Nature. 181: 1199-2000.

Method based on ferric reducing ability: FRAP (Ferric Reducing Ability of Plasma): Benzie, I. I. F.-Strain, J. J. (1996): The ferric reducing ability of plasma (FRAP) as a measuring of “antioxidant power”. The FRAP assay. Annal. Biochem. 239: 70-76.

Trolox Equivalent Antioxidant Capacity, TEAC: Miller, N. J.-Rice-Evans, C.-Davies, M. J.-Gopinathan, V.-Milner, A. (1993): A novel method for measuring antioxidant capacity and its application to monitoring the antioxidant status in premature neonates. Clin. Sci. 84: 407-412.

Antioxidant capacity measurements based on photochemiluminescence:
Antioxidant Capacity Water-Soluble, ACW: Popov, I. N.-Lewin, G. (1994): Photochemiluminescent detection of antiradical activity. 2. Testing nonenzymic water-soluble antioxidants. Free Radic. Biol. Med. 17: 267-271.
Antioxidant Capacity Lipid-Soluble, ACL: Popov, I. N.-Lewin, G. (1996): Photochemiluminescent detection of antiradical activity. 4. Testing of lipid-soluble antioxidants. J. Biochem. Biophys. Methods. 31: 1-8.

Determination of all phenolic components: Singleton V. L., Rossi J. A. (1965): Colorimetry of total phenolics with phosphomolybdic phosphotungstic acid “reagents”. Am. J. Enol. Vitric. 16 p. 144-158.

Determination of proanthocyanidins: Prior, R. L.; Fan, E.; Ji, H.; Howell, A.; Nio, C.; Payne, M. J.; Reed, J. (2010): Multi-laboratory validation of a standard method for quantifying proanthocyanidins in cranberry powders. J. Sci. Food Agric. 90: 1473-1478.

The application of the above methods for sour cherries is described in the following article: Andrea Nemes et al, Determination of Flavonoid and Proanthocyanidin Profile of Hungarian Sour Cherry, Molecules 2018, 23, 3278.

### Determination of Anthocyanins by pH Differential Method:

The measurements were carried out based on Lee, J.-Durst, R. W.-Wrolstad, R. E. (2005): Determination of total monomeric anthocyanin pigment content of fruit juices, beverages, natural colorants, and wines by the pH differential method: Collaborative Study. Journal of AOAC International. 88: 1269-1278, as follows.

From 2 g of the homogenized samples (homogenized fresh sour cherry or sour cherry extract), anthocyanins were extracted with 10 ml of 2% (V/V) HCl-Ethanol solution. A buffer of pH=1.0 was prepared, consisting of 67 ml of HCl solution and 25 ml of KCl solution. The KCl solution was prepared as follows: 1.49 g of KCl was dissolved in 100 ml of distilled water. The HCl solution consisted of the following: 1.7 mL of concentrated HCl was diluted with 100 mL of distilled water. Preparation of the pH=4.5 buffer: 1.64 g of Na-acetate was dissolved in 100 ml of distilled water, then enough concentrated HCl was added to make the solution pH=4.5. The measurement was performed simultaneously at 530 and 700 nm (Amersham Biosciences Ultraspec 2100 pro type spectrophotometer). During the measurement, 400 μl of sample is added to 3600 μl of buffer. During the measurement of the samples, three parallel measurements were performed. The anthocyanin concentration value of the samples was measured in mg cyanidin-3-glucoside equivalents/100 g, based on the fresh sour cherry or the extract.

### Anthocyanin Extraction and UHPLC Analysis

All samples included in the study were homogenized with a Braun Multiprimer (300 Watt) device. The extraction mixture was methanol:water:acetic acid, which was used in a ratio of 25:24:1. The mixing was carried out with a magnetic stirrer (MSH 300, BIOSAN) for 60 minutes at room temperature. After filtration, the extracts were evaporated at 40° C. with a BUCHI ROTAVAPOR R-210 (Switzerland) evaporator. The evaporated samples were redissolved with a 3% formic acid solution. Then, in order to separate the anthocyanins more easily, the fractionation of the sour cherry extracts was pre-conditioned with Supelclean ENVI-18 SPE columns. After purification, the anthocyanin-rich fraction was evaporated.

The samples were analysed with CromasterUltraRs ultra-high-pressure liquid chromatography with a diode-array detector with an automatic sampler and Agilent OpenLAB software, where the stationary phase was: Phenomenex Kinetex 2.6μ, XB.C18, 100A, 100×4.6 mm. The mobile phase: pump A—methanol, pump B—3% formic acid. Detection at 535 nm, and gradient elution was performed 0 min: 15:85, 25 min: 30:70, 30 min: 40:60, 40 min: 50:50. The individual components were identified with HPLC purity standards: Cyanidin-3-glucoside chloride (AppliChem), Malvidin-3-glucoside chloride (AppliChem), Malvidin-3,5-diglucoside chloride (AppliChem), Cyanidin-3-rutinoside chloride (Fluka).

### EXAMPLES

### Example 1

### Preparation:

Sour cherry “ciganymeggy” (Prunus cerasus L.) was harvested by mechanical shaking in the early morning hours and processed immediately after delivery. The stems were removed from the sour cherries, and they were washed, cleaned (the damaged and infected sour cherries were sorted) and drained. After that, the seeds were removed with a fruit-stoner.

### 1st Extraction Cycle:

7.5 L of ethanol:water mixture of 20:80 was poured onto 5 kg of prepared sour cherries. The sour cherries were kept at 25° C. under the filling liquid for 1 hour in a container equipped with a mixer, while stirring 10 times for 1 minute. Meanwhile, the sour cherries were chopped into pieces of about 0.5-0.2 cm.

After that, the mixture was filtered on a (D)<0.1 cm filter. The refractive index of the resulting filtrate was RF=1.5.

The filtrate was evaporated using a rotavapor device, the temperature of the water bath was 40° C., the pressure at the beginning of the evaporation was 150 mbar, which was reduced to 10 mbar.

A sample was taken from the evaporated extract, its viscosity was determined with a Viscolead One type viscometer, and the evaporation was stopped when a viscosity of about 3000 mPa·s was reached.

Thereby, 370 g of extract were obtained.

### Characterization of the Evaporated Extract:

It has a burgundy colour, clearly transparent, not cloudy, smells like sour cherry, has a consistency of honey and is thickly flowing, having a viscosity at 20° C. of 3000 mPa·s, at 40° C. 500 mPa·s and at 5° C. 7050 mPa·s.
pH: 3.8
Density: 1.39 g/cm³
Solubility: dissolves well in water.

The sugar and acid content of the evaporated extract (given in g/100 g of extract) is given in Table 1 below:

TABLE 1

| c(glucose) | c(fructose) | c(saccharose) | c(malic acid) | c(citric acid) |
|---|---|---|---|---|
| g/100 g extr. | g/100 g extr. | g/100 g extr. | g/100 g extr. | g/100 g extr. |
| 24 | 17.5 | 4.1 | 5.2 | 0.2 |

We also determined the antioxidant capacity of the evaporated extract, which is related to the concentration of antioxidant compounds in the concentrate. The table shows the average values of 3 parallel measurements for six batches produced by the method described above. The data are given in the unit of equivalent micrograms/mg extract for the compound corresponding to the measurement method, in Table 2 below.

TABLE 2
Antioxidant capacity measurements (μg/mg extract)

| Treatment | ACL | | | TEAC | | | DPPH | | |
|---|---|---|---|---|---|---|---|---|---|
| 1st | 0.025 | 0.024 | 0.027 | 0.021 | 0.022 | 0.024 | 0.012 | 0.013 | 0.015 |
| extraction | 0.035 | 0.035 | 0.034 | 0.024 | 0.024 | 0.022 | 0.016 | 0.017 | 0.016 |
| | ACW | | | FRAP | | | Total phenols | | |
| | 0.020 | 0.019 | 0.020 | 0.018 | 0.017 | 0.016 | 0.010 | 0.010 | 0.011 |
| | 0.033 | 0.032 | 0.033 | 0.018 | 0.018 | 0.018 | 0.010 | 0.011 | 0.011 |

The proanthocyanidin content of the evaporated extract was also determined, and the data measured for three different batches produced by the method described above are listed in Table 3.

TABLE 3

| | Proanthocyanidin |
|---|---|
| Treatment | (g/100 g extract) |
| 1st | 0.208 |
| extraction | 0.204 |
| | 0.204 |

The above data show that the concentrate obtained after the first extraction does not yet have significant antioxidant capacity and proanthocyanidin content.

The evaporated extract was analysed by UHPLC-MS and the following components were identified: Quinic acid, γ-Aminobutyric acid, Proline, Threonine, Asparagine, Adenine (B4), Pyridoxine (B6), Citric acid, Leucine, Coumaroylquinic acid isomer 1, Coumaroylquinic acid isomer 2, Chlorogenic acid (3-O-Caffeoylquinic acid), Feruloylquinic acid isomer 1, Chryptochlorogenic acid (4-O-Caffeoylquinic acid), Coumaroylquinic acid isomer 3, Feruloylquinic acid isomer 2, Isoquercitrin (Hirsutrin, Quercetin-3-O-glucoside), Abscisic acid, Naringenin

### 2nd Extraction Cycle:

7.8 L of ethanol:water mixture of 50:50 was poured on the residual material remaining after filtration in the 1st extraction step (3.9 kg).

The obtained mixture was kept at 25° C. under the filling liquid for 1 hour, while stirring 10 times for 1 minute. After that, the mixture was filtered on a (D)<0.005 cm filter.

The refractive index of the resulting filtrate was RF=2.5.

The filtrate was combined with the extract obtained in step 1.

The obtained mixture was evaporated using a rotavapor device, the temperature of the water bath was 40° C., the pressure at the beginning of the evaporation was 150 mbar, which was reduced to 10 mbar.

A sample was taken from the evaporated extract, its viscosity was determined with a Viscolead One type viscometer, and the evaporation was stopped when a viscosity of about 3000 mPa·s was reached.

Thereby, 390 g of extract were obtained (the mass increase was 20 g compared to the first extract).

### Characterization of the Evaporated Extract:

It has a deep burgundy colour, clearly transparent, not cloudy, smells like sour cherry, has a consistency of honey and is thickly flowing, its viscosity measured by Viscolead One type viscosimeter at 20° C. is 3010-3070 mPa·s, at 40° C. about 500 mPa·s.
Density: 1.35 g/cm³
Solubility: dissolves well in water and in alcohol.

The sugar and acid content of the evaporated extract is given in Table 4 below:

TABLE 4

| c(glucose) | c(fructose) | c(saccharose) | c(malic acid) | c(citric acid) |
|---|---|---|---|---|
| g/100 g extr. | g/100 g extr. | g/100 g extr. | g/100 g extr. | g/100 g extr. |
| 25.1 | 17.86 | 4.5 | 5.72 | 0.31 |

The data show that the amount of sugars and acids did not change essentially.

The antioxidant capacity and proanthocyanidin content was also determined. The results are given in table 5.

TABLE 5
Antioxidant capacity measurements (μg/mg extract)

| Treatment | ACL | | | TEAC | | | DPPH | | |
|---|---|---|---|---|---|---|---|---|---|
| 2nd | 0.238 | 0.241 | 0.221 | 0.172 | 0.166 | 0.173 | 0.102 | 0.094 | 0.119 |
| extraction | 0.244 | 0.227 | 0.219 | 0.168 | 0.177 | 0.172 | 0.134 | 0.119 | 0.140 |
| | ACW | | | FRAP | | | Total phenols | | |
| | 0.279 | 0.282 | 0.275 | 0.166 | 0.166 | 0.170 | 0.072 | 0.069 | 0.068 |
| | 0.309 | 0.302 | 0.315 | 0.188 | 0.226 | 0.212 | 0.071 | 0.071 | 0.070 |

The proanthocyanidin content of the evaporated extract is given in Table 6.

TABLE 6

| | Proanthocyanidin |
|---|---|
| Treatment | (g/100 g extract) |
| 2nd | 0.395 |
| extraction | 0.420 |
| | 0.386 |

The above data show that that there was a significant increase in the antioxidant capacity (depending on the method, we measured about 10-fold values compared to the first concentrate), and in the proanthocyanidin content as well (it approximately doubled compared to the first concentrate).

The evaporated extract was analysed by UHPLC-MS, and compared to the first evaporated extract, the following additional components were identified:

Asparagine, Leucine, Phenylalanine, Tryptophan, Neochlorogenic acid (5-O-Caffeoylquinic acid), Catechin, Benzyl gentiobioside, Benzyl primeveroside isomer 1, Cyanidin-3-O-sophoroside (Mecocyanin), Tuberonic acid glucoside, Cyanidin-3-O-glucoside (Kuromanin, Asterin, Chrysanthemin), Epicatechin, Benzyl primeveroside isomer 2, Cyanidin-3-O-(2G-glucosyl)-rutinoside, Coumaroylquinic acid isomer 4, Cyanidin-3-O-rutinoside (Keracyanin), Cyanidin-3-O-(2G-xylosyl)-rutinoside, Pelargonidin-3-O-(2G-glucosyl)rutinoside, Cinchonain I isomer 1, Pelargonidin-3-O-rutinoside, Peonidin-3-O-rutinoside, Cyanidin-O-pentoside, Quercetin-O-(hexosyl)rutinoside, Quercetin-O-(hexosyl)hexoside isomer 1, Quercetin-di-O-hexoside, Quercetin-O-rutinoside-O-hexoside, Naringenin chalcone-O-hexoside, Cinchonain I isomer 2, Quercetin-O-(hexosyl)hexoside isomer 2, Cinchonain I isomer 3, Di-O-caffeoylquinic acid, Prunin (Naringenin-7-O-glucoside), Isoquercitrin (Hirsutrin, Quercetin-3-O-glucoside), Rutin (Quercetin-3-O-rutinoside), Dihydroxy(iso)flavone-C-glucoside, Astragalin (Kaempferol-3-O-glucoside), Kaempferol-3-O-rutinoside (Nicotiflorin), Cinchonain I isomer 4, Isorhamnetin-3-O-rutinoside (Narcissin), Abscisic acid, Quercetin-3-O-(4-coumaroyl)glucoside, Naringenin

The mass of the main components was also determined as described above (see: “Anthocyanin extraction and UHPLC analysis”); the main anthocyanins and their amount are as follows: cyanidin-3-0-glucosyl-rutinoside, (11.5 mg/100 g), cyanidin-3-O-rutinoside, (683.1 mg/100 g) and cyanidin-3-O-glucoside (291.4 mg/100 g).

3rd Extraction Cycle: 5 L of ethanol:water mixture of 80:20 was poured on the residual material remaining after filtration in the 2nd extraction step (3.0 kg).

The obtained mixture was kept at 25° C. under the filling liquid for 1 hour, while stirring 10 times for 1 minute. After that, the mixture was filtered on a (D)<0.0005 cm filter.

The refractive index of the resulting filtrate was RF=4.5.

The filtrate was combined with the extract obtained in step 2.

The obtained mixture was evaporated, the temperature of the water bath was 40° C., the pressure at the beginning of the evaporation was 150 mbar, which was reduced to 10 mbar.

A sample was taken from the evaporated extract, its viscosity was determined, and the evaporation was stopped when a viscosity of about 3000 mPa·s was reached.

Thereby, 302 g of extract were obtained (the mass increase was 12 g compared to the first extract).

### Characterization of the Evaporated Extract:

It has a deep burgundy colour, clearly transparent, not cloudy, smells like sour cherry, has a consistency of honey and is thickly flowing, its viscosity is 3070 mPa·s.
pH: 3.5
Density: 1.425 g/cm³
Solubility: dissolves well in water and in alcohol.

The sugar and acid content of the evaporated extract is given in Table 7 below.

TABLE 7

| c(glucose) | c(fructose) | c(saccharose) | c(malic acid) | c(citric acid) |
|---|---|---|---|---|
| g/100 g extr. | g/100 g extr. | g/100 g extr. | g/100 g extr. | g/100 g extr. |
| 25.1 | 18.06 | 4.51 | 5.0 | 0.31 |

The data show that the amount of sugars and acids did not change essentially.

The antioxidant capacity and proanthocyanidin content was also determined. The results are given in tables 8 and 9, respectively.

TABLE 8
Antioxidant capacity measurements (μg/mg extract)

| Treatment | ACL | | | TEAC | | | DPPH | | |
|---|---|---|---|---|---|---|---|---|---|
| Complete | 0.507 | 0.504 | 0.511 | 0.387 | 0.392 | 0.401 | 0.310 | 0.304 | 0.305 |
| extraction | 0.504 | 0.504 | 0.510 | 0.395 | 0.395 | 0.399 | 0.308 | 0.305 | 0.305 |
| | ACW | | | FRAP | | | Total phenols | | |
| | 0.581 | 0.584 | 0.589 | 0.394 | 0.406 | 0.407 | 0.150 | 0.146 | 0.144 |
| | 0.597 | 0.592 | 0.594 | 0.403 | 0.405 | 0.410 | 0.142 | 0.145 | 0.149 |

TABLE 9

| | Proanthocyanidin |
|---|---|
| Treatment | (g/100 g extract) |
| Complete | 1.205 |
| extraction | |

The above data show that there was a further significant increase in the antioxidant capacity (depending on the method it approximately doubled) and in the proanthocyanidin content (it approximately tripled).

The evaporated extract was analysed by UHPLC-MS, and compared to the second evaporated extract, the following additional components were identified: Procyanidin B isomer 1, Procyanidin C isomer 1, Procyanidin C isomer 2, Procyanidin B isomer 2, Catechin, Epicatechin, Procyanidin C isomer 3, Procyanidin B isomer 3 Among the compounds identified, the amount of catechin and epicatechin is the most significant.

In the third concentrate, the main anthocyanins and their amount are as follows: cyanidin-3-O-glucosyl-rutinoside (11.6 mg/100 g), cyanidin-3-O-rutinoside, (685.1 mg/100 g) and cyanidin-3-O-glucoside (291.7 mg/100 g).

### Example 2

### Preparation:

Sour cherry “Újfehért\dot{o}i fürtös” (Prunus cerasus ‘Újfehért\dot{o}i Fürtös’) was harvested by mechanical shaking in the early morning hours. Immediately after delivery, stems were removed from the sour cherries, and they were washed, cleaned (the damaged and infected sour cherries were sorted) and drained. After that, the seeds were removed with a fruit-stoner. The pitted sour cherries were stored in a freezer until use.

### Extraction Cycles:

Three extraction cycles were performed, as described in example 1, with the difference that in the first step 400 ml of ethanol:water mixture of 20:80 was poured onto 200 g of thawed and prepared sour cherries. In the other steps, the solvent mixtures described in example 1 were used, but in a proportionally smaller amount.

The parameters measured to characterize each extract are summarized in Table 10 below. For comparison, we also present the anthocyanin data measured from fresh sour cherries, as well as the sugar and acid data measured from juice pressed from fresh cherries. In the case of fresh cherries, the given amounts refer to 100 g, while in the case of extracts, the anthocyanin data are for 100 g of extract, and the sugar and acid data are for the total extract obtained from 200 g of cherries (the weight of the extract is also shown in the table).

TABLE 10

| | | | after the | after the | after the |
|---|---|---|---|---|---|
| | | Fresh sour | 1st | 2nd | 3rd |
| | | cherry | extraction | extraction | extraction |
| Anthocyanin by total pH | | 220 | 200 | 1418 | 1740 |
| differential method (mg/100 g) | | | | | |
| Purified (mg) | cyanidin-3-O-glucoside | 43 | 21.5 | 258 | 336 |
| | cyanidin-3-O-glucosyl- | 16 | 8 | 120 | 124 |
| | rutinoside | | | | |
| | cyanidin-3-O-rutinoside | 80 | 24 | 300 | 302 |
| Sugar (g/100 g or | glucose | 2.3 | 4.4 | 4.4 | 4.4 |
| g/extract) | fructose | 2.1 | 4.1 | 4.2 | 4.1 |
| | saccharose | 0.012 | 0.009 | 0.024 | 0.024 |
| Acid (g/100 g or | malic acid | 1.2 | 2.4 | 2.0 | 1.9 |
| g/extract) | citric acid | 0.005 | 0.01 | 0.1 | 0.1 |

| | | | | |
|---|---|---|---|---|
| Other | 0.163 | 1.2 | 1.4 | 1.4 |
| Mass of the extract (g) | nr | 25 | 19 | 19 |
| pH of the extract | nr | 3.0 | 3.2 | 3.2 |
| Density of the extract (g/cm3) | nr | 1.2 | 1.38 | 1.48 |
| Viscosity of the extract (Pa • s) | nr | 2800 | 3000 | 3400 |

From the data in the table, it can be concluded that the sugars and acids get already to the extract during the first extraction step, and most of the anthocyanins were dissolved during the second extraction.

### Example 3

### Preparation:

The preparation was carried out as described in Example 2.

1st Extraction Cycle: 400 ml of ethanol:water mixture of 20:80 was poured onto 200 g of prepared sour cherries.

After that, we proceeded as described in the 1st extraction cycle of Example 1.

### 2nd Extraction Cycle:

An 80:20 mixture of ethanol:water was poured onto the material remaining after filtration of the 1st extraction step.

After that, we proceeded as described in the 2nd extraction cycle of Example 1.

The characteristics of the extract obtained with the two extraction cycles are summarized in Table 11 below.

TABLE 11

| | | | | | | | | FRAP | pH diff. |
|---|---|---|---|---|---|---|---|---|---|
| | | | | Mass of the | | | | (mg/100 g) | (mg/100 g) |
| | | Solvent % | | extract | | | ρ | antioxid. | anthocy. |
| | | alcohol | water | (g) | Rf. | pH | (g/cm3) | capacity | quantity |
| Example 3 | 1st extr. | 20 | 80 | 25 | 1.363 | 3.8 | 1.24 | 131.18 | 0.604 |
| | 2nd extr. | 80 | 20 | | | | | | |

### Example 4

### Preparation:

The preparation was carried out as described in Example 2.

### 1st Extraction Cycle:

400 ml of ethanol:water mixture of 20:80 was poured onto 200 g of prepared sour cherries.

After that, we proceeded as described in the 1st extraction cycle of Example 1.

### 2nd Extraction Cycle:

An 50:50 mixture of ethanol:water was poured onto the material remaining after filtration of the 1st extraction step.

After that, we proceeded as described in the 2nd extraction cycle of Example 1.

The characteristics of the extract obtained with the two extraction cycles are summarized in Table 12 below.

TABLE 12

| | | | | | | | | FRAP | pH diff. |
|---|---|---|---|---|---|---|---|---|---|
| | | | | Mass of the | | | | (mg/100 g) | (mg/100 g) |
| | | Solvent % | | extract | | | ρ | antioxid. | anthocy. |
| | | alcohol | water | (g) | Rf. | pH | (g/cm3) | capacity | quantity |
| Example 4 | 1st extr. | 20 | 80 | 13 | 1.43 | 3.8 | 1.38 | 186.03 | 0.714 |
| | 2nd extr. | 50 | 50 | | | | | | |

### Example 5: Shelf Life Test

The extract prepared according to example 1 was stored at room temperature for 4 years and then characterized.

It has a deep burgundy colour, clearly transparent, not cloudy, smells like sour cherry.

State of matter: the extract has crystallized like honey (this indicates the high glucose and fructose content and the low water content, and this is one of the basis of long shelf life). The crystallinity disappears upon gentle heating, but in our experience, moving and shaking the container is sufficient to break the crystallinity (so it is easier to break the crystalline state than in the case of honey).

State of matter after thawing: has the consistency of honey and is thickly flowing.

Solubility: dissolves well in water and in alcohol The sugar and acid content of the evaporated extract is given in Table 13 below.

TABLE 13

| c(glucose) | c(fructose) | c(saccharose) | c(malic acid) | c(citric acid) |
|---|---|---|---|---|
| g/100 g extr. | g/100 g extr. | g/100 g extr. | g/100 g extr. | g/100 g extr. |
| 25.2 | 18.07 | 4.51 | 5.2 | 0.31 |

Proanthocyanidins: 1.11 g/100 g

Main anthocyanins: cyanidin-3-O-glucosyl-rutinoside (9.2 mg/100 g), cyanidin-3-O-rutinoside, (662.2 mg/100 g) and cyanidin-3-O-glucoside (290.2 mg/100 g).

The data prove that the extract essentially preserved its quality and composition for 4 years, and there was no significant change even in the amount of anthocyanins and proanthocyanidins, which are sensitive compounds.

### Example 6: Determination of Germ Count

The determination of the germ count was carried out according to the relevant standards for each sample, and the following results were obtained:

Mesophilic aerobic germ count (according to MSZ EN ISO 4833:2003 standard): 0 CFU/g Yeast and mould count (according to MSZ ISO 7954 standard): 0 CFU/g

### Comparative Example 1: extraction with pure alcohol

Sour cherries prepared as described in example 2 were used. 250 ml of ethanol was poured onto 100 g of prepared sour cherries. The sour cherries were kept under the filling liquid at 25° C. for 1 hour. We have found that the sour cherries hardened, and there was hardly any fragmentation during mixing.

The eluate was evaporated under the parameters described in example 1 to obtain 10 ml of extract.

### Characterization of the Evaporated Extract:

It has a burgundy colour, smells like sour cherry, has a consistency of honey and is thickly flowing, its viscosity measured by Viscolead One type viscosimeter at 20° C. is 3800 mPa·s.

pH: 4.5 g

The sugar and acid content of the evaporated extract is given in Table 14 below (given in g/10 ml of extract).

TABLE 14

| c(glucose) | c(fructose) | c(saccharose) | c(malic acid) | c(citric acid) |
|---|---|---|---|---|
| g/10 ml extr. | g/10 ml extr. | g/10 ml extr. | g/10 ml extr. | g/10 ml extr. |
| 1.7 | 0.81 | 0.0 | 0.2 | not detectable |

Anthocyanin content: 150 mg/10 ml.

In relation to the above, we note the following. The mixture of the sour cherry's own water content and the added alcohol results in a water content of about 25%. Sugars and acids are less soluble in such a solvent, which is supported by the measured lower amounts of sugar and acid, as well as the higher pH value.

In principle, anthocyanins dissolve well in such a solvent, but the amount extracted falls short of the amount obtained with several steps (see e.g. the data provided in example 2), which can be explained by the fact that the permeability of the cell wall decreases due to the high alcohol content, so those substances that would otherwise dissolve well in a solvent of this composition are also extracted to a smaller extent.

### Comparative Example 2: Extraction with Water

Cherries prepared as described in example 2 were used.

200 ml of water was poured onto 100 g of prepared sour cherries. The sour cherries were kept under the filling liquid at 25° C. for 1 hour. By visual observation of the extraction mixture, we found that colouring matter was dissolved, but the initially burgundy coloured solution turned brown, which indicates the decomposition of the colouring matter.

The eluate was evaporated under the parameters described in example 1 to obtain 10 ml of extract.

### Characterization of the Evaporated Extract:

It has a browny-burgundy colour, smells like sour cherry, has a consistency of honey and is thickly flowing, its viscosity measured by Viscolead One type viscosimeter at 20° C. is 3000 mPa·s.

pH: 3.0 g

Density: 1.35 g/cm3

Solubility: dissolves well in water

The sugar and acid content of the evaporated extract is given in Table 15 below (given in g/10 ml of extract).

TABLE 15

| c(glucose) | c(fructose) | c(saccharose) | c(malic acid) | c(citric acid) |
|---|---|---|---|---|
| g/10 ml extr. | g/10 ml extr. | g/10 ml extr. | g/10 ml extr. | g/10 ml extr. |
| 3.7 | 2.1 | 0.012 | 1.2 | 0.005 |

Anthocyanin content in 10 ml extract: 37 mg.

In relation to the above, we note the following. The amount of sugar and acid that can be dissolved in theory is practically dissolved with water. The dissolution of anthocyanins and colouring matter also begins, but their (partial) decomposition is indicated on the one hand by the colour change and also by the measured low anthocyanin content.

### Comparative Example 3: Extraction with Solvents of Increasing Alcohol Content and Evaporation of Individual Filtrates by Themselves

We proceeded according to Example 2, with the difference that the eluate obtained after the 2nd and 3rd extractions was evaporated alone, without adding the concentrate from the previous steps.

We found that the material turned brown when evaporated, which indicates decomposition.

This observation supports the fact that the presence of the first fraction extracted with a solvent mixture with a high water content is important for stability not only in the case of the final product, but also during the process. Even though in this case the materials extracted in the first and optionally the second extraction step go through several evaporation steps, we do not experience significant decomposition. On the other hand, without the presence of the first fraction, the evaporation of the later fractions leads to significant decomposition, i.e. loss of active ingredients (browning is an obvious sign of this).

### Example 7: Use of the Sour Cherry Extract According to the Invention in Case of Non-Alcoholic Fatty Liver Disease in a Rabbit Model

### Background:

Non-alcoholic fatty liver disease (NAFLD) is the most common chronic liver disease and occurs in 25% of the population worldwide. The rapid increase in prevalence in recent years parallels the prevalence of obesity and metabolic syndrome. The most common causes of NAFLD are improper eating habits, hyperlipidaemia and obesity resulting from a high-fat and low-fibre diet. However, fatty liver can develop, for example, in connection with intestinal bypass operations, abdominal surgery, extensive small bowel resections, inflammatory bowel diseases, Crohn's disease, gluten-sensitive enteropathy, but starvation and protein deficiency can also lead to fatty liver. Oestrogen-containing drugs, oral contraceptives, hormone therapy drugs, etc. can similarly contribute to liver damage and also increase the risk of gallstone formation. Convincing scientific research has proven that the transsulfuration pathway (TSP) plays a central role in metabolic syndromes. It also seems to be confirmed that the transsulfuration pathway (TSP) is related to the production of the antioxidant glutathione (GSH) and the signal molecule hydrogen sulphide (H₂S), and both are related to the pathogenesis of NAFLD.

The large number of patients with liver disease related to NASH (non-alcoholic steatohepatitis) and the emerging pharmacological treatment options indicate an urgent need for valid and reproducible biomarkers of the development and progression of the disease, as well as bioactive components acting on molecular targets that can be incorporated into the diet.

Based on the following preclinical studies, the sour cherry extract according to the invention—presumably due to its high concentration of anthocyanins—appears to be suitable for stimulating the regeneration of liver cells, which can be assessed by the decrease of liver necroenzymes in the plasma. The extract has also been proven to lower cholesterol levels.

### Rabbit Model Animals

The number of the ethical permission of the experiment: 4/2022/DEMAB.

Before the experiment, the 20 New Zealand White rabbits did not receive any special pre-treatment, water and normal laboratory food were available to them ad libitum. After the arrival of the animals, a 2-week acclimatization period followed. At the starting point of the experiment, the animals were randomly divided into two groups with the same number of animals: one group received standard rabbit food for the entire duration of the experiment, and the ill animal model was created in the other group. “Atherogenic” food, enriched with 1% cholesterol and 1% triglyceride, was intermittently given for 3 weeks, then standard food for 1 week, repeating this eight times, so it took 32 weeks to develop the animal model. After 32 weeks, both groups were further divided into 2 groups, thus forming 4 groups. The treatment with sour cherry extract according to the invention was started after that.

The classification of the groups is as follows:
Group I: healthy control group, received normal rabbit food throughout the experiment; (C)
Group II: received sour cherry extract treatment in addition to normal food; (Control+anthocyanin)
Group III: hypercholesterolemic (HC) animals kept on an atherogenic diet for the entire duration of the experiment, they did not receive any other treatment;
Group IV: in addition to the atherogenic diet, they received sour cherry extract treatment. (HC+anthocyanin)

The animals participating in the treatment received the sour cherry extract according to the invention dissolved in 250 ml of drinking water for 12 weeks after the 32nd week at a dose of 9 g/kg body weight (this ensured the anthocyanin intake). The body weight of the animals was measured weekly.

At the end of the experiment, the planned tests were carried out, and then the animals were over-anesthetized with ketamine-xylazine (150/10 mg/kg intramuscular injection). After thoracotomy, the aorta was removed and its endothelium-dependent vasorelaxation was examined, the organs were removed (in formalin and liquid nitrogen), and a stool sample was also taken for later tests.

### 7.1. Clinical Blood Tests

Plasma was obtained from blood taken noninvasively into blood collection tubes containing EDTA anticoagulant. The analysis was performed with a Roche cobas c 311 analyser. The analyser complies with the protection requirements laid down in the in vitro diagnostic (IVD) directive 98/79/EC. (In addition, it was manufactured and tested according to the following international standards: IEC 61010-1:2001; IEC 61010-2-010:2003; IEC 61010-2-081:2001; IEC 61010-2-101:2002; UL 61010-1:2001; CAN/CSA C22.2 No. 61010-1-04; EN 61326-2-6:2006.) The Roche/Hitachi cobas c 311 analyser is a software-controlled, automatic system designed for clinical chemical analysis. It can be used to perform—in vitro qualitative and quantitative—analysis using a wide range of analytical tests. It performs photometric tests and ion-selective electrode measurements. It is extremely suitable for meeting small and medium operating load demands.

Blood samples were taken, and blood tests were performed at 4 different times in order to monitor the changes in the effect of the treatment over time.

### 7.1.1. Cholesterol Level

Cholesterol is a fat-like compound that forms blood fats (lipids) in addition to triglycerides. Most of the cholesterol in the body is produced by the liver, but a significant amount can also enter the body through food. An excessive amount of cholesterol present in the blood is a high risk factor for the development of heart and coronary artery diseases, as it is deposited in the vessel walls and forms plaques, which can lead to atherosclerosis or, by blocking the narrowed artery, to the development of thrombosis, heart attack, or stroke.

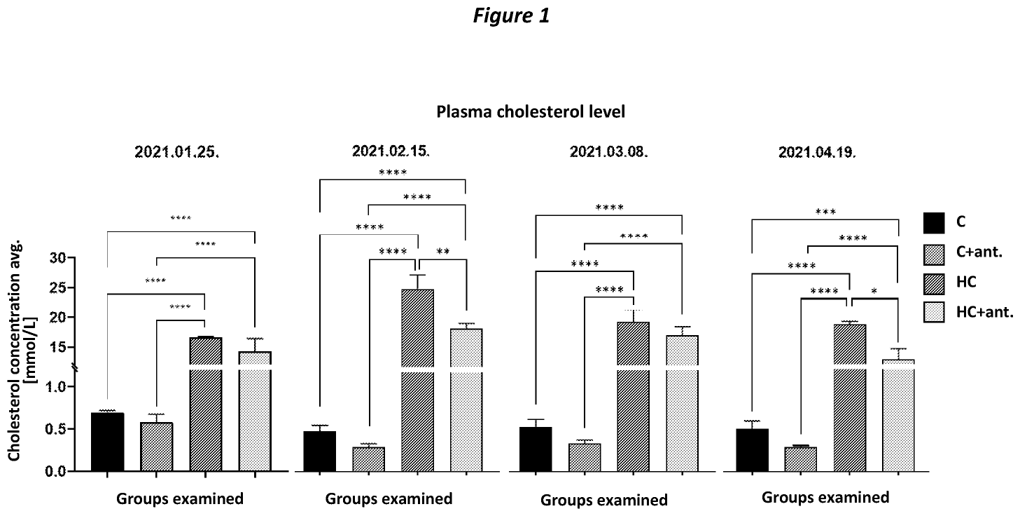

The results obtained in the tests are presented in FIG. 1.

The experiment proves that by feeding the sour cherry extract, we were able to reduce the extremely high cholesterol level caused by the atherogenic diet.

### 7.1.2. Liver Enzyme Levels

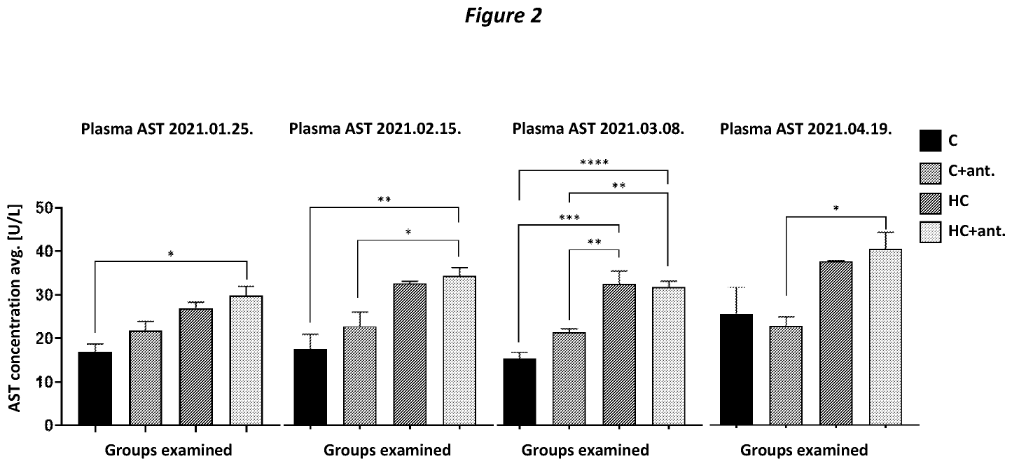

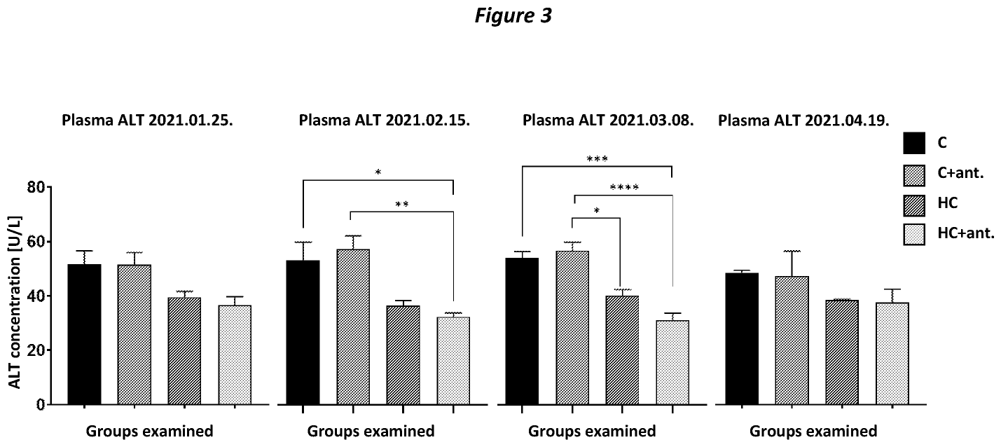

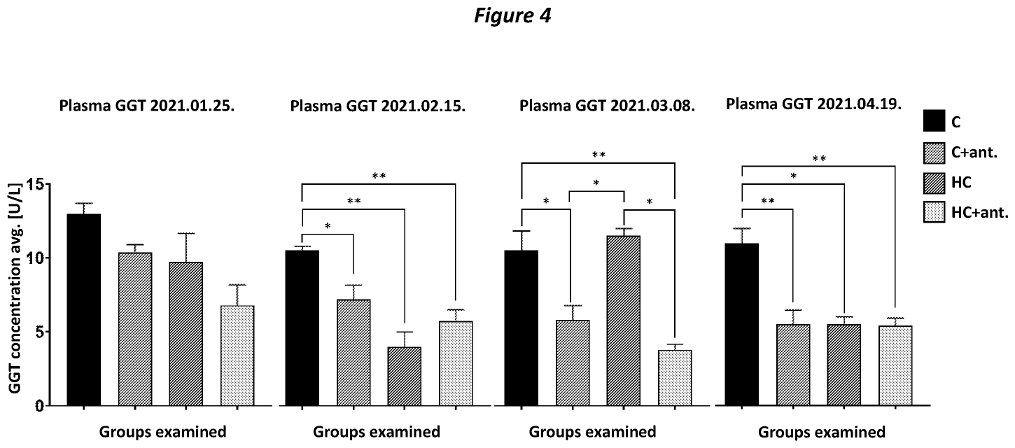

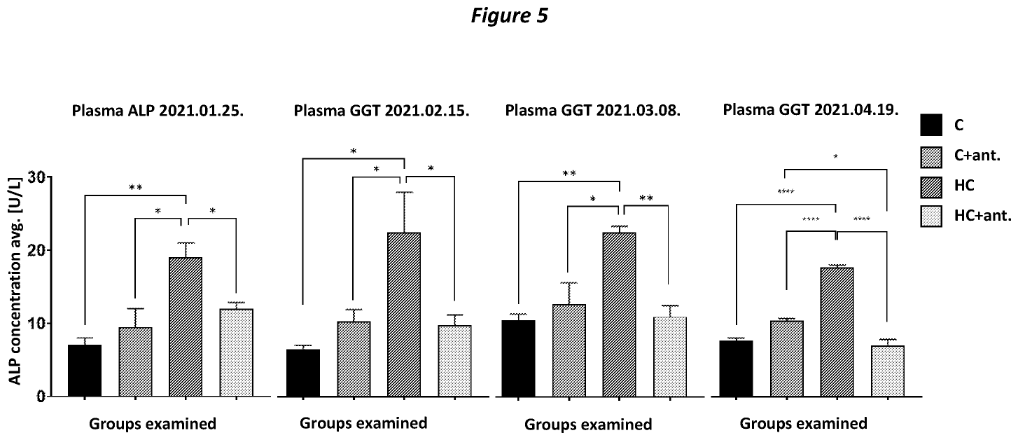

The levels of the following liver enzymes were determined from the blood taken from the animals: AST (aspartate aminotransaminase, also known as glutamate oxaloacetate transaminase, abbreviated as GOT), ALT (alanine aminotransferase, also known as glutamate-pyruvate transaminase, abbreviated as GPT), GGT (gamma-glutamyl transferase) and ALP (alkaline phosphatase). The results are shown in FIGS. 2, 3, 4 and 5, respectively.

Regarding the evaluation of the results, we note the following.

Enzymes that indicate hepatocyte damage or cell death include transaminases and cholestatic liver enzymes, which are released from the cells or their synthesis is induced as a result of damaging factors affecting the liver. An increase in transaminase enzymes [glutamate-oxaloacetate (GOT) and glutamate-pyruvate transaminase (GPT)] indicates liver cell damage, while an increase in alkaline phosphatase (ALP) indicates cholestasis. A low platelet count and a GOT/GPT ratio >1 also reflect advanced fibrosis.

The mechanism of ALP increase is primarily the de novo synthesis in hepatocytes under the influence of bile acids, rather than impaired excretion. A pathological increase in ALP is mainly caused by diseases affecting the extra- and intrahepatic bile ducts, which are the consequences of obstruction in the common or intrahepatic bile duct.

Gamma-glutamyl transferase (GGT) is present in significant amounts in the liver (on the apical membrane of biliary epithelial cells and hepatocytes) but is not present in bone tissue. It is a sensitive predictor of liver damage, and the best predictor of liver-related mortality.

It can be seen that these enzymes are characteristic of different pathological conditions, and their extreme elevated levels together indicates the pathological physiological processes of the liver.

The significant decrease in ALP and GGT enzyme concentrations shows that the sour cherry extract according to the invention can stimulate the regeneration of liver cells.

### 7.2. Gene Expression

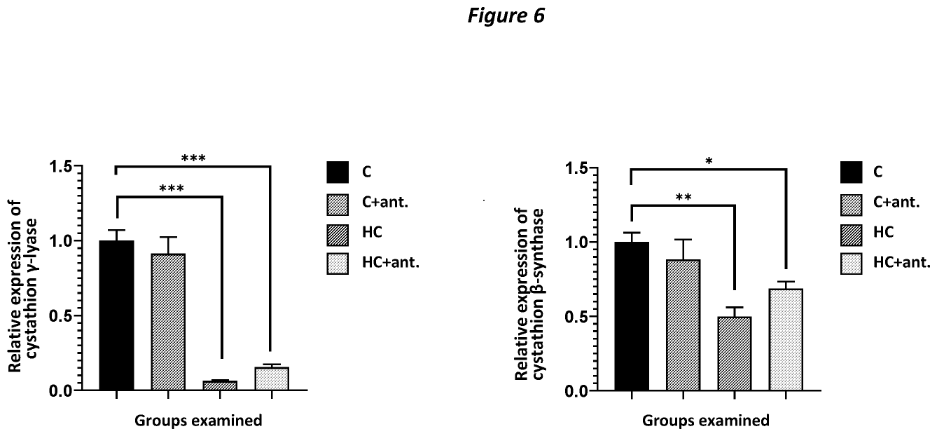

The gene expression of the two enzymes of the transsulfuration pathway (cystathion-γ-lyase and cystathion-β-synthase), which are key elements to our study, as well as the cytokines confirming the inflammatory status, were examined using the TaqMan qPCR (quantitative polymerase chain reaction) method in liver tissue. The results are presented in FIG. 6.

The conversion of homocysteine to cysteine through the intermediate cystathion plays a key role in sulphur metabolism and the redox environment of cells. This pathway is the only pathway for the endogenous biosynthesis of cysteine. The first step is catalysed by the vitamin B6-dependent cystathionine β-synthase (CBS) enzyme, using homocysteine and serine as substrates, forming cystathion in a condensation reaction. The second step is a hydrolysation reaction catalysed by the vitamin B6-dependent cystathionine γ-lyase (CSE) using cystathionine as a substrate to produce cysteine and α-ketobuirate. Both are highly expressed in the liver and to a lesser extent in various other tissues. The high expression of these enzymes greatly ensures the liver's transsulfuration capacity.

The experimental results show that the sour cherry extract according to the invention is able to improve the expression levels decreased by the atherogenic diet.

### INDUSTRIAL APPLICABILITY OF THE INVENTION

With the method according to the invention, the valuable bioactive substances can be effectively extracted from sour cherries, so sour cherry extracts with a high polyphenol and anthocyanin concentration and complex composition can be produced.

The complex extract according to the invention can be used as food, dietary supplement, food for special medical purposes or as a medical device on its own, but it is also suitable for further processing, for example it can be added to food intended for human consumption, functional food or animal feed, or certain active ingredients/groups of active ingredients can be extracted from it in pure form (e.g. by supercritical extraction, chromatography).

The complex extract according to the invention retains its chemical stability for years even without the use of added preservatives or additional preservation processes.

1-21. (canceled)

22. Complex extract made from sour cherry, characterized in that it
has a viscosity at 20° C. of about 2500-3800 mPa·s
has a density of 1.30-1.85 g/cm³
dissolves well in water and in alcohol,
is substantially free of fibres, and
has a pH of 2.8-4.0.

23. The complex extract according to claim 22, further characterized in that it has a deep burgundy colour and sour cherry smell.

24. The complex extract according to claim 22, further characterized in that it has a water-soluble antioxidant capacity (ACW) of about 0.21-0.6 μg/mg extract, the total amount of phenolic compounds is about 0.06-0.2 μg/mg extract.

25. The complex extract according to claim 22, further characterized in that it has a proanthocyanidin content of about 0.3-1.3 g/100 g extract.

26. The complex extract according to claim 22, further characterized in that it comprises the following components: cyanidin-3-O-rutinoside, cyanidin-3-O-glucoside and cyanidin-3-O-glucosyl-rutinoside,
their total amount is about 700 mg-2500 mg/100 g extract,
and their relative ratio is as follows:
cyanidin-3-O-glucosyl-rutinoside: about 0.7-1.3 mass %
cyanidin-3-O-rutinoside: about 65-73 mass %
cyanidin-3-O-glucoside: about 27-32 mass %.

27. The complex extract according to claim 22, further characterized in that it comprises:
glucose, fructose and saccharose, wherein the relative ratio of the individual components within
their total amount is:
glucose: 35-60 mass %,
fructose: 30-55 mass %,
saccharose: 5-15 mass %, and
malic acid and citric acid, wherein the relative ratio of the individual components within their total amount is:
malic acid: 85-98 mass %,
citric acid: 2-15 mass %.

28. Process for the preparation of a complex sour cherry extract according to claim 22, the process comprising
a first extraction cycle, wherein
sour cherry is extracted with a water:ethanol solvent mixture,
the extraction mixture is separated into its phases, thus obtaining an eluate and a phase comprising residual solid material,
the eluate is evaporated, thus obtaining a first concentrate,
one or more further extraction cycle(s), wherein
the fraction comprising residual solid material, obtained in the previous extraction cycle, is extracted with a water:ethanol solvent mixture, whose ethanol content is higher than that of the water-ethanol mixture used in the previous extraction step,
the extraction mixture is separated into its phases, thus obtaining an eluate and a phase comprising residual solid material,
the thus obtained eluate is combined with the concentrate obtained in the evaporation step of the previous extraction cycle, and this mixture is evaporated thus obtaining another concentrate from the current extraction cycle,
wherein
the first extraction is carried out using a 90:10-65:35 water:ethanol solvent mixture, and
the last extraction is carried out using a 50:50-0:100 water:ethanol solvent mixture, wherein the complex extract is obtained as the concentrate from the last extraction cycle.

29. The process according to claim 28, characterized in that two extraction cycles are carried out:
the first extraction is carried out using a 90:10-65:35 water:ethanol solvent mixture, and
as a further extraction, a second extraction is carried out using a 50:50-30:70 water:ethanol solvent mixture.

30. The process according to claim 28, characterized in that two extraction cycles are carried out:
the first extraction is carried out using a 90:10-65:35 water:ethanol solvent mixture, and
as a further extraction, a second extraction is carried out using a 35:65-0:100 water:ethanol solvent mixture.

31. The process according to claim 28, characterized in that
the first extraction is carried out using a 90:10-65:35 water:ethanol solvent mixture, and as further extraction,
a second extraction is carried out using a 65:35-35:65 water:ethanol solvent mixture, and
a third extraction is also carried out using a 35:65-0:100 water:ethanol solvent mixture.

32. The process according to claim 31, characterized in that
the first extraction is carried out using a 85:15-75:25 water:ethanol solvent mixture, and
the second extraction is carried out using a 60:40-40:60 water:ethanol solvent mixture, and
the third extraction is carried out using a 30:70-0:100 water:ethanol solvent mixture.

33. The process according to claim 28, characterized in that
the first extraction step is carried out within a time period of at most 24 hours, and optionally
the further extraction steps are carried out within a time period of at most 12 hours.

34. The process according to claim 28, characterized in that the separation of the extraction mixture into its phases is carried out by filtration, and the aperture size of the filter used in the last filtration step is at most 0.01 cm.

35. Composition comprising a complex extract according to claim 22.

36. The composition according to claim 35, which is a dietary supplement, food for special medical purposes or medical device.

37. The composition according to claim 36, comprising the complex extract formulated in capsules.

38. The composition according to claim 35, which is a food composition intended for human consumption, comprising said complex extract and other food ingredients.

39. The composition according to claim 35, which is a feed composition for animals, comprising said complex extract and other feed ingredients.

40. A method of preventing tooth decay; preventing or reducing inflammations, e.g. colitis, prostatitis; reducing pain and muscle pain caused by osteoarthritis; regulating carbohydrate metabolism, including lowering blood sugar; reducing the feeling of hunger, preventing obesity; preventing non-alcoholic fatty liver disease or promoting liver regeneration processes in case of an already established disease; preventing cancer, or inhibiting of the growth of tumour cells in the case of an already established tumour, wherein the complex extract according to claim 1 is administered to a person in need thereof in an effective amount.

41. A method according to claim 40, which is for preventing non-alcoholic fatty liver disease or promoting liver regeneration processes in case of an already established disease.
